(19) United States
(12) Patent Application Publication
(10) Pub. No.: US 2026/0081011 A1
(43) Pub. Date: Mar. 19, 2026

(54) COMPLIANCE MONITORING SYSTEM

(71) Applicant: IMAM ABDULRAHMAN BIN FAISAL UNIVERSITY, Dammam (SA)

(72) Inventors: Khaled Fikry SALAMA, Dammam (SA); Hana Mohammed ALHARTHI, Dammam (SA); Noor Maher FELEMBAN, Dammam (SA)

(73) Assignee: IMAM ABDULRAHMAN BIN FAISAL UNIVERSITY, Dammam (SA)

(21) Appl. No.: 18/889,087

(22) Filed: Sep. 18, 2024

(51) Int. Cl. G16H 40/60; G16H 40/20

(52) U.S. Cl. CPC G16H 40/60; G16H 40/20

(57) ABSTRACT

A compliance monitoring system including a face mask with a first sensor, a gown with a second sensor, a face shield, or an eye shield with a third sensor, and a pair of gloves with a fourth sensor and a fifth sensor individually disposed on a righthand and a lefthand of the pair of gloves. The compliance monitoring system further includes a processor having circuitry and a memory. The circuitry includes instructions configured to receive input signals from the sensor reader and send an alarm notification when the face mask, the gown, the face shield or eye shield, and the pair of gloves are donned or doffed by a user in an order that is either consistent or inconsistent with a predetermined sequence saved in the memory of the processor.

### BACKGROUND

### Technical Field

The present disclosure is directed to systems and methods for monitoring personal protective equipment protocols, specifically to a system and a method designed to monitor the donning and doffing of personal protective equipment (PPE) in healthcare and related environments to ensure compliance with established safety protocols.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

Healthcare-associated infections (HCAIs) continue to cause morbidity and mortality in most countries, despite increases in preventative measures taken to limit infection spread in healthcare environments. Healthcare facilities commonly require staff to wear specific types of PPE, such as gloves, gowns, masks, and eye protection, when interacting with patients, performing surgeries, or handling contaminated materials. Proper donning (putting on) and doffing (taking off) procedures are critical components of PPE protocols, designed to minimize exposure to infectious agents and ensure the safety of both the healthcare workers and patients. Despite the prevalence of these protocols in healthcare settings, there are still significant challenges associated with ensuring consistent compliance with these protocols. Manual monitoring of PPE practices can be subject to human error and non-compliance with these protocols can go unnoticed, resulting in health safety concerns. Incorrect donning and doffing of PPE can further lead to cross-contamination, increased risk of infection transmission, and other serious consequences, highlighting the importance of strict adherence to established protocols.

Conventional solutions to monitor compliance with PPE protocols typically involve training programs, supervision, and periodic compliance checks. For example, manual checklists may be used to track adherence to PPE in healthcare environments, however, this method, and others commonly utilized, rely heavily on human oversight, which can be inconsistent and inefficient. Specifically, manual monitoring methods may not provide immediate feedback to the user, which is required for correcting non-compliant behavior at the moment it occurs. Furthermore, manual monitoring methods can be labor-intensive, diverting healthcare staff from other critical tasks. Additionally, existing systems generally lack the capability to track individual compliance data over time, which is important for identifying patterns of non-compliance and targeting specific training needs.

US20190043337A1 describes a system that utilizes a wristband to monitor PPE compliance based on predefined sequences of movements, particularly in hospital settings. The proposed system uses motion sensors integrated into a wristband that communicates with a stationary controller. This reference does not implement dedicated sensors on each item of PPE that communicate with a central processor, ensuring compliance based on the donning or doffing sequence of each item individually.

CN 111387617A describes a protective suit with a wear detection device that transmits signals to an external module to indicate compliance with wearing protocols. The proposed suit focuses on detecting correct wearing through radio frequency signals but lacks real-time feedback mechanisms for correcting actions. This reference does not implement dedicated sensors on each item of PPE that communicate with a central processor, ensuring compliance based on the donning or doffing sequence of each item individually.

CN 112989960A describes a method and a system for monitoring putting-on and taking-off behaviors of protective articles based on a computer vision technology, relying on visual data processing to enforce compliance. This approach might suffer from limitations in environments with poor visibility or when the protective articles are not visually distinct. This reference does not implement dedicated sensors on each item of PPE that communicate with a central processor, ensuring compliance based on the donning or doffing sequence of each item individually.

Each of the cited references suffers from one or more drawbacks hindering their success in ensuring proper donning and doffing protocol. Accordingly, it is one object of the present disclosure to provide real-time, automated monitoring of PPE protocol compliance. The present disclosure seeks to address these issues by introducing a comprehensive compliance monitoring system equipped with unique identifiers and sensors for each PPE item, linked to a processor that can immediately detect and respond to compliance issues, to improve safety standards, reduce the risk of infection, and streamline the monitoring of PPE practices in healthcare settings.

### SUMMARY

In an exemplary embodiment, a compliance monitoring system is described. The compliance monitoring system comprises a face mask comprising a first sensor placed on a middle section of the face mask, wherein the middle section is configured to be positioned near a mouth of a user. The compliance monitoring system also comprises a gown comprising a second sensor disposed on a neckline of the gown. The compliance monitoring system further comprises a face shield or an eye shield comprising a third sensor disposed on an upper forehead section of the face shield or a nose bridge of the eye shield. The compliance monitoring system further comprises a pair of gloves comprising a fourth sensor and a fifth sensor individually disposed on a righthand and a lefthand of the pair of gloves. The compliance monitoring system further comprises a processor having circuitry and a memory. The circuitry comprises instructions configured to receive input signals from a sensor reader, upon the sensor reader scanning the first through the fifth sensors, and send an alarm notification when the face mask, the gown, the face shield or eye shield, and the pair of gloves are donned or doffed by a user in an order that is either consistent or inconsistent with a predetermined sequence saved in the memory of the processor.

In some embodiments, each sensor comprises a unique identifier selected from the group consisting of a serial number, a barcode, an integrated chip, or a Radio Frequency Identification (RFID) tag. In one embodiment, each unique identifier is relayed by a signal to the processor when an associated sensor is read by the sensor reader. In this embodiment, the sensor reader is in wireless communication with the processor.

In some embodiments, each unique identifier is individually paired to the face mask, the gown, the face shield or eye shield, and the pair of gloves to obtain a paired unique identifier. Each sensor with the paired unique identifier is activated when scanned by a sensor reader, in wireless communication with the processor, as the user dons the face mask, the gown, the face shield or eye shield, or the pair of gloves.

In some embodiments, the memory is configured to store doffing and donning compliance data for a plurality of users. The circuitry is further configured to determine a compliance rate of the plurality of users based on the doffing and donning compliance data. In some embodiments, each sensor further comprises a color sensor, a thermal sensor, or a QR code sensor reader. The color sensor, a thermal sensor, or a QR code sensor reader may relay information such as PPE selection or whether the PPE was donned or doffed by the user. In this embodiment, this information is relayed to the processor by a sensor reader, which is wirelessly connected to the processor. The sensor reader scans each color sensor, thermal sensor, and/or QR code, then transmits a signal to the processor relaying said information to the processor, to be used in determining whether a correct selection of PPE and order of donning/doffing is performed by the user.

In some embodiments, the circuitry is further configured to determine a correct pairing of the face mask, the gown, the face shield or eye shield, and the pair of gloves by a user.

In some embodiments, each user is associated with an individual user identifier. The memory of the processor is further configured to store a plurality of the individual user identifiers. The individual user identifier may be associated with an individual sensor reader or a fingerprint of the user. The compliance monitoring system may further comprise a fingerprint sensor reader, which sends the individual user identifier to the processer. In one embodiment, the sensor reader may be included in an identification badge, subsequently transmitting the individual user identifier when each sensor is read by the sensor reader.

In some embodiments, the predetermined sequence is individually coded for a procedure type based on a selected compliance standard.

In some embodiments, the system further comprises a controlling mechanism. The system is disposed in a first room, used as a donning and/or doffing area, and the controlling mechanism is configured to stop the user from proceeding into a next room from the first room when the order is inconsistent with the predetermined sequence.

In some embodiments, the circuitry is further configured to alert the user to stop and correct the order to be consistent with the predetermined sequence before proceeding when the order is inconsistent with the predetermined sequence. In another embodiment, the circuitry may transmit a signal to the processor to relay the alert to the user, such as by a flashing light, a sound, or operating the controlling mechanism.

In some embodiments, the first through fifth sensors are RFID tags.

In some embodiments, the circuitry is configured to calculate a monthly average of noncompliance for the user from the compliance data.

In some embodiments, when the monthly average of noncompliance exceeds a predetermined limit, a notification is created to alert the user an additional training program is needed.

In some embodiments, the system further comprises a display device. The display device is configured to show an image of the face mask, the gown, the face shield or eye shield, and the pair of gloves in the order consistent with the predetermined sequence.

In some embodiments, the display device is configured to show a video of a model user donning or doffing the face mask, the gown, the face shield or eye shield, and the pair of gloves in the order consistent with a predetermined technique and the predetermined sequence.

In some embodiments, the system further comprises a timer and a speaker. Herein, the timer is set for a predetermined amount of time for handwashing when required by the predetermined sequence. Further, the speaker is configured to alert the user when the predetermined amount of time for handwashing is complete.

In some embodiments, the memory is further configured to store a cleaning history such that when the system is placed in a clean room for sterile compounding, the system is configured to alert the user that a cleaning is due, consistent with a predetermined clean room standard.

In some embodiments, the system further comprises a unique cleaning identifier associated with a sensor. The sensor is selected from the group consisting of a serial number, a barcode, an integrated chip, or an RFID tag. When the cleaning is complete, the circuitry is configured to receive an input signal from the sensor reader which scans the sensor, creating a cleaning log. Further, the memory is configured to store a plurality of the cleaning logs.

In another exemplary embodiment, a method of monitoring a user donning or doffing PPE with the compliance monitoring system is described. The method comprises identifying the user by an individual user identifier. The method further comprises selecting the predetermined sequence associated with a procedure type. The method further comprises instructing the user to don or doff the face mask, the gown, the face shield or eye shield, and the pair of gloves in a stepwise order based on the predetermined sequence. The method further comprises sending an alarm notification when the order is inconsistent or consistent with the predetermined sequence.

In some embodiments, the method further comprises individually scanning the first through fifth sensor with a sensor reader before the user dons or doffs the face mask, the gown, the face shield or eye shield, and the pair of gloves.

The foregoing general description of the illustrative embodiments and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 is a schematic diagram of a compliance monitoring system, according to certain embodiments.

FIG. 2 is a schematic diagram illustrating an exemplary implementation of the compliance monitoring system within a healthcare setting, according to certain embodiments.

FIG. 3 is a schematic diagram illustrating a sequence of interactions between sensors associated with personal protective equipment items and a processor of the compliance monitoring system, according to certain embodiments.

FIG. 4 is a flowchart depicting steps involved in a process of monitoring donning procedures within the compliance monitoring system, according to certain embodiments.

FIG. 5 is a flowchart depicting steps involved in a process of monitoring doffing procedures within the compliance monitoring system, according to certain embodiments.

FIG. 6 is a flowchart depicting steps involved in a method of monitoring a user donning or doffing personal protective equipment with the compliance monitoring system, according to certain embodiments.

FIG. 7 is an exemplary graph of incidence of healthcare-associated infections (HAIs) by specialty and distribution of HAI types by specialty, according to certain embodiments.

FIG. 8 is an illustration of a non-limiting example of details of computing hardware used in the computing system, according to certain embodiments.

FIG. 9 is an exemplary schematic diagram of a data processing system used within the computing system, according to certain embodiments.

FIG. 10 is an exemplary schematic diagram of a processor used with the computing system, according to certain embodiments.

FIG. 11 is an illustration of a non-limiting example of distributed components which may share processing with the controller, according to certain embodiments.

### DETAILED DESCRIPTION

In the drawings, like reference numerals designate identical or corresponding parts throughout the several views. Further, as used herein, the words “a,” “an” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

Aspects of this disclosure are directed to a compliance monitoring system and a method of monitoring a user donning or doffing personal protective equipment with the compliance monitoring system, that comprises a set of uniquely identifiable sensors attached to various PPE items, specifically, a face mask, a gown, a face shield or eye shield, and each glove of a pair of gloves. Each sensor is read by a sensor reader, the sensor reader being capable of sending signals to a central processor, which is programmed to track and verify a sequence in which the PPE items are worn or removed. The system alerts the user of any deviations from a predetermined sequence, thus ensuring proper compliance. This system is particularly useful in healthcare settings to reduce the risk of HAIs and improve overall safety practices.

The compliance monitoring system 100 (hereinafter, sometimes, simply referred to as “system 100” without any limitations) is designed to ensure adherence to personal protective equipment (PPE) protocols within healthcare and related environments. The compliance monitoring system 100 is configured to monitor the donning and doffing of PPE, such as gloves, gowns, masks, and eye or face shields. This monitoring is performed through a series of sensors uniquely placed on each PPE item, which are read by a sensor reader, the sensor reader then communicating a signal providing information such as a PPE identifier, a time stamp of when the PPE was selected, a time stamp of when the PPE was donned, and the like, with a processor 102 having a circuitry 104 and a memory 106. The processor 102 of the compliance monitoring system 100 further comprises a circuitry which is programmed with a predetermined sequence of donning and doffing, which serves as a benchmark for correct PPE usage. When a deviation from this predetermined sequence is detected, the compliance monitoring system 100 is configured to send an alarm notification, such as flashing light, a sound, or a similar alert, thereby informing the user of the error, and prompting corrective action. Thereby, the compliance monitoring system 100 facilitates immediate compliance with established safety protocols and contributes to the overall infection control efforts within the healthcare facility and the like.

In one embodiment, the processor of the compliance monitoring system 100 is placed in a room used for sterilization prior to entering a surgical room, a patient room, or the like. In another embodiment, the processor is located in an ante room preceding the clean room of a sterile compounding lab.

The compliance monitoring system 100 comprises a face mask 110. The face mask 110 comprises a first sensor 114 placed on a middle section 112 of the face mask 110. Herein, the middle section 112 is configured to be positioned near a mouth of a user. The placement of the first sensor 114 on the middle section 112 ensures proximity to the mouth of the user, which is required for monitoring proper PPE usage, especially in healthcare settings where protection against respiratory infections is important. The middle section 112 of the face mask 110 is thus configured to align with and cover the mouth area, allowing the first sensor 114 to be centrally located on the mask. In one embodiment, the first sensor comprises a thermal sensor which may be read by a sensor reader to detect whether the mask has been placed on the user and whether the mask is in the correct place on the user. The first sensor 114 is read by a sensor reader, the sensor reader generating an input signal indicative of the donning and doffing actions performed by the user with respect to the face mask 110. The input signals are transmitted to the processor 102 of the compliance monitoring system 100, by the sensor reader, which then assesses whether the face mask 110 has been applied or removed in accordance with the specific sequence, or technique, stored in the memory 106 therein (as discussed later in more detail). This arrangement ensures that the face mask 110 is used effectively to reduce the risk of contamination and enhance the overall compliance with health and safety standards.

In one embodiment, the user has a sensor reader (preferably a RFID sensor reader) that scans the first sensor on the face mask. In another embodiment, another sensor may be placed on a container that organizes the different types of face masks in the donning/doffing area, enabling a signal to be captured when the user chooses the specific type of mask that must be worn. Individual sensors, each with a unique identifier, may be used for each distinct type of container having PPE, such as to distinguish a standard surgical mask from a N95 mask. In yet another embodiment, the user scans a container holding the proper face mask, then scans the first sensor located on the face mask when the user has donned the face mask.

The RFID sensor reader, when present on or in contact with a user, is preferably a short-range sensor reader operating at frequencies ranging from 125 kHz to 2.45 GHz. The RFID sensor reader is capable of sensor reader an RFID tag that is place in contact with the RFID sensor reader or in close proximity to the RFID sensor reader, e.g., at a distance of within 10 cm, preferably within 5 cm such as the SYNOTAG short range RFID sensor readers in 125 KHz, 13.56 MHz and UHF ISO-18000-6B or 6C EPC Gen 2.

In one embodiment, when the sensor reader scans a sensor, a signal is transmitted to the processor. When the signal is received by the processor, a recording of the unique identifier displaying the user, an identifier displaying the PPE selected and the PPE donned, each with an associated time stamp generated to associate a time in which the user selected and/or donned the PPE. The unique identifiers and time stamps recorded by the processer are relayed to the circuitry, which then compares the selection of PPE and the time stamps to a pre-determined sequence, to determine whether the user has selected the proper PPE and donned the selected PPE in accordance with the pre-determined sequence.

In one embodiment, the sensor reader used by the user is in wired or wireless communication with the processor. The sensor reader may be a sensor reader chip integrated into an identification badge. In another embodiment, the sensor reader may be a portable electronic QR code scanner, such as a cellular phone, a tablet, and the like. In yet another embodiment, the sensor reader may be a handheld barcode scanner.

The procedure or process for doffing and donning PPE is preferably carried out with a system in which, as noted above, each component of the PPE has a separate RFID tag permitting identification and association with unique data. Preferably, the RFID sensor reader is embedded in an identification badge and/or access badge which is carried by a user, for example, on a neckband, as a wallet card or in the form of a fob. When donning PPE, the user places the RFID tag from each component of PPE in proximity to or in contact with the RFID sensor reader that is embedded in the badge. The resulting signal is a “donning signal” which may be recorded in memory and is time stamped. At the end of the donning session, the sequence of donning based on the unique identifier for each component of the PPE and an associated donning timestamp may be processed. A timestamp each donning event permits determination whether the sequence of donning meets requirements. Similarly, when doffing PPE, a user contacts or brings in close proximity to the RFID sensor reader, each piece of PPE after it is removed and before it is discarded. Similar timestamp and saving in memory permits determination whether a user has donned and/or doffed PPE in a correct sequence.

The compliance monitoring system 100 further comprises a gown 120. The gown 120 comprises a second sensor 124 disposed on a neckline 122 of the gown 120. The placement of the second sensor 124 on the neckline 122 ensures that it is positioned to allow for monitoring the proper use of the gown 120, especially in healthcare settings in which maintaining a sterile barrier from a body of the user using the gown is required. The neckline 122 of the gown 120 is designed to align with the area around the user's neck. In one embodiment, the second sensor 124 comprises a thermal sensor which may be read by a sensor reader to detect whether the gown 120 has been placed on the user and whether the gown is in the correct place on the user. In one embodiment, the second sensor 124 is read by a sensor reader, thus generating an input signal indicative of the donning and doffing actions performed by the user with respect to the gown 120. The input signal may transmit information such as the time stamp at which the proper gown was selected and donned/doffed, in addition to the type of gown selected. The input signals are transmitted to the processor 102 of the compliance monitoring system 100, which then assesses whether the gown 120 has been applied or removed in accordance with the specific sequence stored in the memory 106 therein (as discussed later in more detail). This arrangement ensures that the gown 120 is used correctly to reduce the risk of contamination and enhance the overall compliance with health and safety standards.

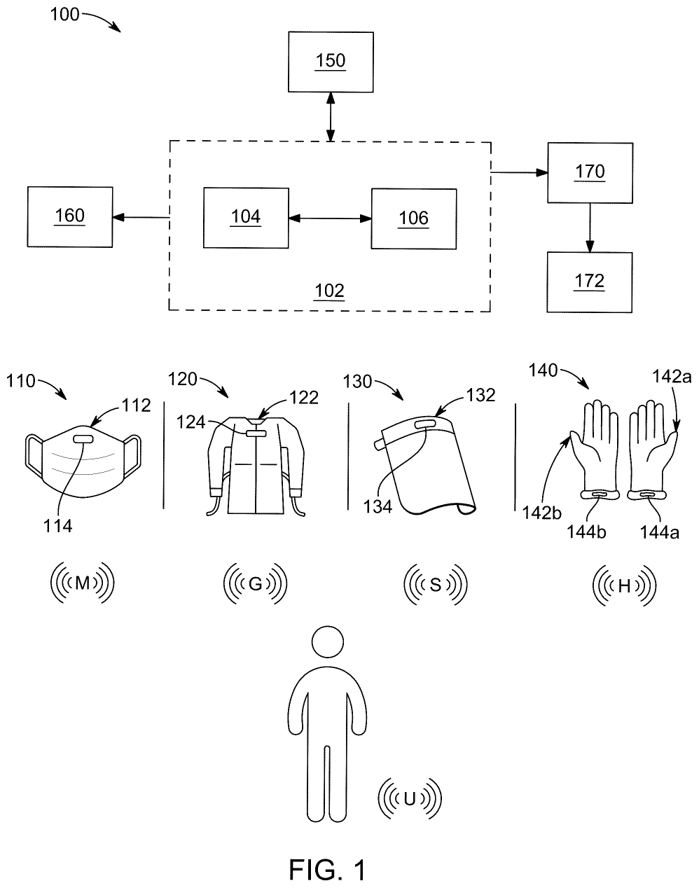

The compliance monitoring system 100 further comprises a face shield or an eye shield, herein the face shield being depicted in FIG. 1 and represented by reference numeral 130; however, it may be replaced with the eye shield without departing from the spirit and the scope of the present disclosure. The description in the foregoing paragraphs may refer to the face shield 130 and the eye shield 130, generally, interchangeably, without any limitation. The face shield or eye shield 130 comprises a third sensor 134, which is disposed on an upper forehead section 132 of the face shield 130 or on a nose bridge (also not depicted in FIG. 1 but may generally be referred to as the nose bridge 132 in the description, without any limitations) of the eye shield 130. The placement of the third sensor 134 on the upper forehead section 132 or the nose bridge 132 ensures its proximity to areas for monitoring proper PPE usage and donning technique, particularly in protecting against airborne particles or splashes that could transmit infections. In one embodiment, the third sensor 134 comprises a thermal sensor which may be read by a sensor reader to detect whether the face shield 130 has been placed on the user and whether the face shield is in the correct place on the user. In one embodiment, the third sensor 134 is read by a sensor reader, thus generating an input signal indicative of the donning and doffing actions performed by the user with respect to the face shield. The input signal may transmit information such as the time stamp at which the proper face shield was selected and donned/doffed, in addition to the type of face shield selected. The input signals are then transmitted from the sensor reader to the processor 102 of the compliance monitoring system 100, which then assesses if the face shield or eye shield 130 has been applied or removed in accordance with the specific sequence stored in the memory 106 therein (as discussed later in more detail). This arrangement ensures that the face shield or eye shield 130 is used correctly to reduce the risk of contamination and enhance the overall compliance with health and safety standards.

The compliance monitoring system 100 further comprises a pair of gloves 140. The pair of gloves 140 comprises a fourth sensor 144a and a fifth sensor 144b, which are individually disposed on a righthand glove 142a and a lefthand glove 142b of the pair of gloves 140, respectively. The individual placement of the fourth sensor 144a on the righthand glove 142a and the fifth sensor 144b on the lefthand glove 142b ensures precise monitoring of use of each glove, which is required in healthcare settings where proper glove usage is essential for preventing cross-contamination and maintaining sterile conditions. In one embodiment, the fourth sensor 144a on the righthand glove 142a and the fifth sensor 144b on the lefthand glove 142b each comprises a thermal sensor which may be read by a sensor reader to detect whether the fourth sensor 144a on the righthand glove 142a and the fifth sensor 144b on the lefthand glove 142b has been placed on the user and whether the fourth sensor 144a on the righthand glove 142a and the fifth sensor 144b on the lefthand glove 142b are in the correct place on the user. In one embodiment, the fourth sensor 144a on the righthand glove 142a and the fifth sensor 144b on the lefthand glove 142b are each individually read by a sensor reader, thus generating input signals indicative of the donning and doffing actions performed by the user with respect to the pair of gloves. The input signal may transmit information such as the time stamp at which the proper pair of gloves were selected and donned/doffed, in addition to the type of gloves selected. The input signals are transmitted by the sensor reader to the processor 102 of the compliance monitoring system 100, which then evaluates whether each glove has been applied or removed following the specific sequence stored in the memory 106 therein. This arrangement ensures that both gloves 142a, 142b in the pair of gloves 140 are used correctly to reduce the risk of contamination and enhance the overall compliance with health and safety standards.

It may be noted that each of the face mask 110, the gown 120, the face shield or eye shield 130, and the pair of gloves 140, as implemented for purposes of the present disclosure, may be disposable PPE items as commonly available in the market and utilized in healthcare facilities. In another embodiment, the PPE may be reusable PPE, subject to sterilization after use by a user. The corresponding sensors, namely, the first sensor 114 for the face mask 110, the second sensor 124 for the gown 120, the third sensor 134 for the face shield or eye shield 130, and the fourth sensor 144a and fifth sensor 144b for the pair of gloves 140, may be taken off one article of PPE and put on another of the same type (i.e., from one surgical mask to another). In another embodiment, the sensors are disposable.

The circuitry 104 within the compliance monitoring system 100 is configured to receive input signals from the sensor reader upon scanning the first through the fifth sensors associated with various PPE items. As discussed, the first sensor 114 is attached to the face mask 110, the second sensor 124 is attached to the gown 120, the third sensor 134 is attached to the face shield or eye shield 130, and the fourth sensor 144a and the fifth sensor 144b are attached to the righthand glove 142a and the lefthand glove 142b of the pair of gloves 140, respectively. The circuitry 104 facilitates the processing of these input signals, which indicate the donning and doffing actions of the PPE by the user. The sensor reader is configured to relay input signals comprising data regarding which sensor is read, and when, along with which user is donning/doffing the PPE. This information reflects whether its corresponding PPE item has been put on or taken off in a predetermined sequence and/or with a predetermined technique. Upon receiving these input signals, the circuitry 104 evaluates the input signals to determine if the actions comply with the required protocols, as discussed in the proceeding paragraphs.

In the compliance monitoring system 100, each sensor may be selected from a group consisting of a serial number, a barcode, an integrated chip, or an RFID tag. In one embodiment, each sensor is associated with a unique identifier. As shown in the exemplary configuration, the first sensor 114 on the face mask 110 is assigned with a unique identifier represented as ‘M’, the second sensor 124 on the gown 120 is assigned with a unique identifier represented as ‘G’, the third sensor 134 on the face shield or eye shield 130 is assigned with a unique identifier represented as ‘S’, and the fourth sensor 144a and fifth sensor 144b on the righthand glove 142a and lefthand glove 142b of the pair of gloves 140 is assigned with a unique identifier represented as ‘H’. This assignment enables each of these PPE items to be individually recognized and monitored by the compliance monitoring system 100 when the sensor is read by the sensor reader in wireless communication with the processor 102. That is, upon reading each sensor, the sensor reader facilitates wireless communication to processor 102 of the compliance monitoring system 100 by transmitting information associated the unique identifier of the sensor read and the timing in which the sensor was read. This wireless communication enables real-time transmission of data regarding the donning and doffing of each PPE item. As each sensor is read by the sensor reader, i.e., either the application or removal of the PPE to which it is attached, the sensor reader sends the input signal to the processor 102. This input signal, encoded with the unique identifier and other information such as the corresponding time stamp at which the sensor was read, ensures that the processor 102 can accurately determine which PPE item was involved in the action, as well as the time and sequence of the action.

In particular, each unique identifier is individually paired to a sensor, with each sensor being individually paired to the face mask 110, the gown 120, the face shield or eye shield 130, and the pair of gloves 140 to obtain a paired unique identifier, thus identifying the specific PPE upon reading the sensor. This pairing process involves assigning a unique identifier to each sensor specifically linked to its respective PPE item. This arrangement ensures that each PPE item is distinctly recognizable and traceable within the compliance monitoring system 100. Once each unique identifier is paired with its corresponding PPE item, each sensor with the paired unique identifier is read by the sensor reader when the PPE is first selected and when the user dons the face mask 110, the gown 120, the face shield or eye shield 130, or the pair of gloves 140. That is, the first sensor 114 on the face mask 110, the second sensor 124 on the gown 120, the third sensor 134 on the face shield or eye shield 130, and the fourth sensor 144a and fifth sensor 144b on the right and left hands of the pair of gloves 140 respectively, are configured to activate when the user reads the sensor located on the respective PPE item. Each sensor with the paired unique identifier is then deactivated when the sensor reader is used to read each sensor associated with the PPE as the user doffs said PPE. Therefore, each sensor may be used in a plurality of donning and doffing cycles with the same user or with varying users, with the activation and deactivation occurring during several, individual donning and doffing events as the sensor is read by the sensor reader. The processor is configured to understand when a sensor is deemed activated or deactivated, based on the time stamp of the first input signal sent by the sensor reader when scanning an article of PPE and each subsequent signal sent by the sensor reader when scanning the same article of PPE.

In a preferred embodiment, the sensor is an RFID tag. The RFID tag may be an active or a passive RFID tag. A passive RFID tag is a battery-less tag typically used in smaller-scale operations and tracking systems. Because a passive tag has no internal power source, it is instead powered by the energy transmitted from an RFID scanner or sensor reader. An active RFID tag comes equipped with its own battery and internal transmitter in the form of a beacon or transponder. This means that an active tag is able to continuously transmit signals to an RFID sensor reader up to 150 meters away, depending on frequency levels. In a preferred embodiment, the sensor is a passive RFID tag, enabling the user to indicate when the sensor is read as the PPE is donned/doffed. The choice of RFID tags for the first sensor 114 on the face mask 110, the second sensor 124 on the gown 120, the third sensor 134 on the face shield or eye shield 130, and the fourth sensor 144a and fifth sensor 144b on the righthand glove 142a and lefthand glove 142b of the pair of gloves 140, respectively, enhances functionality and reliability of the compliance monitoring system 100. Furthermore, RFID tags can store a significant amount of data, which can include not only the unique identifier but also additional information such as the type of PPE, and the specific compliance standards it meets. Thus, the incorporation of RFID tags within the compliance monitoring system 100 contributes to broader management capabilities that improve operational efficiency and adherence to safety protocols within healthcare environments.

Further, in the compliance monitoring system 100, the circuitry 104 comprises instructions configured to send an alarm notification when the face mask 110, the gown 120, the face shield or eye shield 130, and the pair of gloves 140 are donned or doffed by a user in an order that is either consistent or inconsistent with a predetermined sequence saved in the memory 106 of the processor 102. That is, the circuitry 104 manages alarm notifications based on the adherence to the predetermined sequence, and technique, of donning or doffing the PPE items. As discussed, the predetermined sequence is stored within the memory 106 of the processor 102. The processor 102 is configured to handle the input signals transmitted from the sensor reader when the sensor reader reads and/or scans the first sensor 114 on the face mask 110, the second sensor 124 on the gown 120, the third sensor 134 on the face shield or eye shield 130, and the fourth sensor 144a and fifth sensor 144b on the righthand glove 142a and lefthand glove 142b of the pair of gloves 140, respectively. When a user dons or doffs the face mask 110, the gown 120, the face shield or eye shield 130, and the pair of gloves 140, the sensor reader reads/scans the sensor, thus sending an input signal to the processor 102. The circuitry 104 then processes the input signals to determine whether the sequence and technique in which the PPE items were donned or doffed aligns with the predetermined sequence and technique saved in the memory 106. In one embodiment, if the sequence followed by the user is consistent with the predetermined sequence, the compliance monitoring system 100 may simply continue monitoring without interruption. In another embodiment, if the sequence is inconsistent, indicating either a premature or incorrect order of donning or doffing, the circuitry 104 is programmed to trigger the alarm notification. This alarm notification alerts the user to the deviation, allowing for immediate correction, and it ensures continuous adherence to the established protocols. Thereby, the compliance monitoring system 100 effectively reduces the risk of contamination and enhances safety measures within healthcare environments.

In one embodiment, the predetermined sequence is individually coded for a procedure type based on a selected compliance standard. This coding may be stored in the memory 106 of the processor 102 and is tailored to meet specific safety requirements of different medical procedures or healthcare settings. Each sequence is designed to ensure that the use of PPE item, comprising the face mask 110, the gown 120, the face shield or eye shield 130, and the pair of gloves 140, aligns with the standard practices and protocols. This process may involve defining a sequence for each type of PPE application that corresponds to a particular medical procedure. For instance, the sequence for donning PPE for a surgical procedure might differ from that required for handling a patient diagnosed with an infectious disease and placed in a quarantine zone. Each sequence specifies the order in which items like the face mask 110, the gown 120, the face shield or eye shield 130, and the gloves 140 are to be selected, donned/doffed, and/or worn. The processor 102 utilizes this individually coded sequence to monitor and verify the correct application of PPE by healthcare workers. When a user begins the process of donning or doffing PPE, the sensor reader scans/reads each sensor associated with each PPE item, to send signals to the processor 102. The processor 102 utilizes the predetermined, procedure-specific sequences stored in the memory 106 to determine whether the PPE is being used correctly according to the specified standard for that particular procedure. If a deviation from the predetermined sequence is detected, the processor 102 triggers the alarm notification, alerting the user to adjust their practice to align with the required standards. This ensures precise adherence to safety standards, as required for protecting healthcare workers and patients from potential risks associated with improper PPE usage.

In the compliance monitoring system 100, the circuitry 104 is further configured to alert the user to stop and correct the order to be consistent with the predetermined sequence before proceeding when the order is inconsistent with the predetermined sequence. In some embodiments, this alert may be a sound, a flashing light, or a controlling mechanism that bars entry into the next room from the room in which the user is donning/doffing the PPE. As discussed, the predetermined sequence, as stored in the memory 106 of the processor 102, may be tailored to various procedure types based on selected compliance standards, ensuring that each step in the sequence is important for maintaining optimal safety and hygiene practices. When a user begins to don or doff the PPE item(s), including the face mask 110, the gown 120, the face shield or eye shield 130, and the pair of gloves 140, the corresponding sensors attached to each PPE item (the first sensor 114, the second sensor 124, the third sensor 134, the fourth sensor 144a, and the fifth sensor 144b are read by the sensor reader which transmits the input signals to the processor 102. The input signals indicate the order in which the PPE items are being worn or removed based on time stamps of when the sensor was read/scanned by the sensor reader. The circuitry 104 processes the input signals to determine if the sequence followed by the user matches the predetermined sequence coded for that specific medical procedure or setting. If the circuitry 104 detects that the order in which the PPE is donned or doffed deviates from the predetermined sequence, it immediately triggers the alarm notification. The alarm notification is adapted to instruct the user to stop and correct their actions before proceeding. Thereby, the compliance monitoring system 100 ensures that the user adjusts the sequence of donning or doffing the PPE items to be consistent with the prescribed order.

In an embodiment, each sensor further comprises a color sensor and/or a QR code. The color sensors and/or QR codes are included in the first sensor 114 on the face mask 110, the second sensor 124 on the gown 120, the third sensor 134 on the face shield or eye shield 130, and the fourth sensor 144a and fifth sensor 144b on the righthand glove 142a and lefthand glove 142b of the pair of gloves 140. The color sensors and/or QR codes may be configured to relay additional information to the processor, when read by the sensor reader, if the PPE items are correctly matched and worn by identifying color-coded PPE and/or QR coded PPE items. This capability ensures that PPE items also conform to specific requirements that may be color-dependent, such as in environments where different colors signify different levels of protection. The color sensors and QR codes may further be configured to relay a signal to the processor, when read by the sensor reader, to detect if the PPE item is properly selected from other PPE items of the same class. For example, a color sensor may aid the processor in detecting that a N95 mask is donned, instead of a surgical mask. Each color sensor enables the transmission of color-specific data from the color sensors to the processor 102, when the sensor reader reads the color sensors. When a PPE item is donned, the color sensor is read by the sensor reader, the sensor reader relaying a signal to the processor that signal being used to detect if the color of the item matches predefined criteria stored in the memory 106 of the processor 102. If a discrepancy or an incorrect match is identified, the processor 102 may initiate corrective measures through alarm notifications or the like, thus aiding in maintaining high standards of safety and compliance within healthcare settings.

In the compliance monitoring system 100, the circuitry 104 is further configured to determine a correct pairing of the face mask 110, the gown 120, the face shield or eye shield 130, and the pair of gloves 140 by a user. This functionality ensures that the PPE items, in addition to being worn consistently with the predetermined sequence, are also the appropriate ones designated for specific medical procedures or environments. The process of determining the correct pairing involves analyzing input signals received from the sensor reader by reading/scanning each sensor assigned to each PPE item. These input signals send data to the circuitry 104 when a user dons the PPE. This data may be selected from time stamps, unique identifiers identifying specific PPE articles, individual user identifiers, and the like. The circuitry 104 then compares this data against a predetermined set of criteria stored in the memory 106 to verify if each PPE item matches the requirements for the particular medical or healthcare setting. These criteria may include factors like the type, size, and even specific color codes that certain procedures may require. If the circuitry 104 detects that any of the PPE items do not match the required specifications, it may trigger an alarm notification which informs the user of the mismatch, prompting them to correct the error by selecting the appropriate PPE item. This helps in maintaining strict adherence to safety protocols and enhances the overall compliance within healthcare environments. This further allows for the compliance monitoring system to be used in a wide variety of settings due to the ability to individualize the system to the specific needs of the setting.

In some embodiments, the compliance monitoring system 100 may further include motion sensors, which are utilized to detect movement and orientation changes. Motion sensors come in two basic types: active and passive. A motion detector that is considered passive, like a passive infrared (PIR) motion sensor, does not emit any kind of pulse or energy. Instead, passive sensors monitor the area looking for changes in heat and vibrations. Specifically, PIR sensors register infrared energy associated with body heat and detect the resulting change in temperature in the area surrounding the sensor. This type of sensor can detect both heat and movement in its surrounding area, creating a protective “grid.” If the sensor picks up rapidly changing infrared energy levels, it may trigger an alarm. In contrast, an active motion sensor constantly emits light or energy, looking for changes in how the reflection of light travels back to the sensor. An example of an active motion sensor is an area reflective sensor. Area reflective sensors constantly emit pulses of infrared light from a light-emitting diode and then use the reflection from these rays to measure the distance to a person or object. These motion sensors can be integrated within the PPE items to track body movements during the donning and doffing procedures. If the motion sensors detect irregular movements or deviations from the predetermined sequence stored in the memory 106, these motion sensors are configured to trigger alerts to the processor 102. In one embodiment, an active motion sensor detects whether a user attempts to move from one room, in which the processor is located, and the donning/doffing occurs, to the next, before completing the donning/doffing procedure. In another embodiment, the motion sensors are used to track the movements of the user, to ensure that proper technique is utilized when donning specific articles of PPE. For example, the motion sensors may be configured to detect whether the user dons their gloves according to proper sterile technique when the processor is used in the ante room of a sterile compounding lab. This functionality ensures that PPE is donned and doffed in the correct order, and with the proper technique, thereby maintaining safety and compliance with established protocols.

Further, in the compliance monitoring system 100, each user is associated with an individual user identifier, represented by “U”. Such individual user identifier “U” may be used to uniquely distinguish each healthcare professional or staff member who interacts with the compliance monitoring system 100. Herein, the memory 106 of the processor 102 is configured to store a plurality of these individual user identifiers “U”. This allows the compliance monitoring system 100 to maintain a database of all users, tracking their interactions with the PPE items, including the face mask 110, the gown 120, the face shield or eye shield 130, and the pair of gloves 140. Each interaction is logged with reference to the individual user identifier “U,” enabling the compliance monitoring system 100 to monitor adherence to PPE protocols at an individual level. This enables the compliance monitoring system 100 to provide personalized feedback to users based on their specific needs and historical compliance data. For instance, if a particular user repeatedly fails to follow the correct PPE donning sequence, the compliance monitoring system 100 can flag this behavior against a profile of that user. This, in turn, provides a mechanism for tracking and improving PPE usage practices across healthcare facilities, thereby improving overall safety and compliance with established protocols. In one embodiment, this individual user identifier is transmitted by the sensor reader to the processor using the fingerprint of the user. In this embodiment, the user places their fingerprint on a sensor reader capable of reading individual fingerprints, the fingerprint being associated with the user's individual user identifier. In another embodiment, the individual user identifier is transmitted by a sensor reader contained with an identification badge. In this embodiment, the identification badge is linked to a specific user and individual user identifier. Thus, when the identification badge is used to read any of the sensors of the PPE, the individual user identifier is sent alongside the unique identifier and other data provided by reading/scanning the sensor of the PPE.

In an implementation, the memory 106 is configured to store doffing and donning compliance data for a plurality of users. This allows the compliance monitoring system 100 to collect and archive detailed records of how each user interacts with the PPE item, such as the face mask 110, the gown 120, the face shield or eye shield 130, and the pair of gloves 140. This compliance data comprises each instance of PPE being donned and doffed, timestamped, and linked to the individual user identifiers. Moreover, the circuitry 104 is further configured to determine a compliance rate of the plurality of users based on the doffing and donning compliance data. The compliance rate may be calculated by analyzing the frequency and consistency with which users adhere to the prescribed PPE protocols, including the correct order of donning and doffing, as determined by the predetermined sequences stored in the memory 106. This allows healthcare facilities to monitor overall adherence to safety protocols and provides a quantitative measure of how effectively PPE guidelines are being followed across the facility.

In some embodiments, the circuitry 104 is configured to calculate a monthly average of noncompliance for the user from the compliance data. This calculation may involve aggregating instances where a user fails to adhere to the prescribed PPE protocols, such as not following the predetermined sequence for donning or doffing the face mask 110, the gown 120, the face shield or eye shield 130, and the pair of gloves 140, and then averaging these instances over the course of each month. Further, when the monthly average of noncompliance exceeds a predetermined limit, a notification is created to alert the user an additional training program is needed. That is, when any of the user's monthly average of noncompliance exceeds a predetermined limit, which is set based on organizational standards or safety requirements, the circuitry 104 is programmed to generate a notification, to alert the user that their noncompliance rate has exceeded acceptable levels and that an additional training program is necessary to address these deficiencies. This alert may be sent to the user the next time they attempt to utilize the processor in scanning PPE. In another embodiment, this alert may be sent to a method of contact associated with the individual user identifier such as a telephone number or an email. In yet another embodiment, the processor may be associated with a mobile application or an electronic medical record (EMR) system, thus notifying the user of their deficiency on their mobile device or on a computer on which they have accessed the EMR. The processor may be further configured to “lock out” a user until the mandatory training is completed. In this embodiment, the user will be alerted when they scan the first sensor with the sensor reader that they are unable to continue donning/doffing the PPE using the compliance monitoring system. The processor may be further configured to allow a separate individual to monitor the donning/doffing procedures of the user to bypass the compliance monitoring system's alert. After monitoring the user, the separate individual may use the sensor reader to transmit their individual user identifier to the processor, confirming they monitored the locked-out user while the user donned/doffed the PPE and that said donning/doffing was performed in compliance with the predetermined sequence associated with the procedure type. Further, by identifying users who specifically need additional training, the compliance monitoring system 100 ensures that resources are allocated effectively, and that training is focused on individuals who require it most, thus optimizing the impact of training programs.

In some embodiments, the compliance monitoring system 100 further comprises a controlling mechanism 150, which may be installed within a first room. Such setup may be implemented in facilities where maintaining a sterile environment or ensuring procedural compliance is critical, such as in surgical units, isolation rooms, sterile compounding labs, or testing laboratories. The controlling mechanism 150 may be configured to prevent users from proceeding into a next room from the first room if the order of donning or doffing personal PPE items is inconsistent with the predetermined sequence, as stored in the memory 106. In one embodiment, the controlling mechanism may be an automatic lock on a door separating the first room from the next. As discussed, the predetermined sequence comprises the correct application and removal of items such as the face mask 110, the gown 120, the face shield or eye shield 130, and the pair of gloves 140. When a user attempts to transition from one room to another, the processor communicates with the controlling mechanism 150. If the processor 102 detects a deviation from the predetermined sequence, the circuitry 104 triggers the controlling mechanism 150 to activate a barrier, or a locking system, which effectively restricts the user's movement, thereby preventing access to the next room until the user corrects the sequence error and complies with the required PPE protocol. Thus, by integrating the controlling mechanism 150 within the compliance monitoring system 100, the healthcare facilities can achieve a higher level of control and safety, ensuring that the environments are kept secure and that all personnel comply with safety standards before proceeding to sensitive areas.

In another embodiment, the controlling mechanism may be a motion sensor or a thermal sensor reader. When the controlling mechanism is a motion sensor, the processor receives a signal from the motion sensor detecting whether the user is attempting to enter the threshold the controlling mechanism controls before the donning/doffing procedure is complete. In another embodiment, the controlling mechanism is a thermal sensor reader. In this embodiment, the thermal sensor reader reads/scans a thermal sensor contained with the first to fifth sensors of the PPE. The thermal sensor reader is configured to read the thermal sensors on each of the first to fifth sensors associated with the PPE, thus confirming that the user has donned/doffed properly. If the thermal sensor reader detects that a thermal sensor is misplaced, the processor will alert the user that they cannot proceed forward without correction.

In an embodiment, the memory 106 is further configured to store a cleaning history when the system 100 is placed in a clean room for sterile compounding. This allows for maintaining the integrity of sterile environment of the clean room by ensuring that all necessary cleaning procedures are carried out consistently and in accordance with predetermined clean room standards. The memory 106 may log each cleaning event, recording data such as the date, time, and cleaning procedure performed. Further, the system 100 is configured to alert the user that a cleaning is due consistent with a predetermined clean room standard. These clean room standards may include, for example, information about specific intervals at which cleaning must be performed and the type of cleaning needed. When the compliance monitoring system 100 determines that the interval since the last recorded cleaning meets or exceeds the prescribed threshold, it triggers an alert to inform the user that a cleaning is due. Such alert ensures that the cleaning protocols are executed before any delay in room cleanliness could lead to contamination.

In some embodiments, the compliance monitoring system 100 further comprises cleaning sensors selected from the group consisting of a serial number, a barcode, an integrated chip, or an RFID tag. The cleaning sensors are associated with an individual cleaning identifier such that each cleaning sensor may be associated with a specific cleaning process. When the cleaning is complete, the processor 102 is configured to receive an input signal from the sensor reader upon the sensor reader reading/scanning the cleaning sensor. The input signal received by the processor uses the individual cleaning identifier to create a cleaning log. This cleaning log may include data such as the date, time, nature of the cleaning performed, and the identifier of the cleaning device or process used. The circuitry is configured to determine whether the proper cleaning was performed based on the cleaning that is due and/or the predetermined clean room standards. Further, the memory 106 is configured to store a plurality of the cleaning logs. This maintains a historical record of all cleaning activities for compliance with regulatory standards, which may require proof of regular cleaning to prevent contamination, especially in environments like clean rooms. The stored cleaning logs can be accessed to verify adherence to cleaning protocols, analyze the frequency and effectiveness of the cleaning procedures, and ensure that all activities are carried out in line with the established clean room standards.

Further, the compliance monitoring system 100 may include a display device 160. The display device 160 is configured to show an image of the face mask 110, the gown 120, the face shield or eye shield 130, and the pair of gloves 140 in the order consistent with the predetermined sequence. In particular, the display device 160 is configured to show a video of a model user donning or doffing the face mask 110, the gown 120, the face shield or eye shield 130, and the pair of gloves 140 in the order consistent with the predetermined sequence and with a technique consistent with a predetermined donning or doffing technique. This way the display device 160 visually guides users by showing images, or videos, of the PPE items in the order they should be donned or doffed. This visual aid is designed to assist users in following the correct protocol for donning and doffing PPE, thereby reducing errors, and enhancing compliance with safety standards. Additionally, in some embodiments, the display device 160 may be adapted based on real-time data received from the sensors associated with each PPE item. If a user deviates from the predetermined sequence, the display device 160 may update to indicate the error and visually guide the user back to the correct point in the sequence. Thus, the display device 160 serves as an educational tool and a reminder to users, ensuring that each step of the procedure is performed correctly.

In some embodiments, the compliance monitoring system 100 comprises a timer 170 and a speaker 172, which may be utilized for enforcing hygiene protocols, particularly handwashing. Herein, the timer 170 is set for a predetermined amount of time for handwashing when required by the predetermined sequence. Such predetermined amount of time aligns with healthcare standards that specify how long handwashing should last to effectively remove potential contaminants and reduce the spread of infections. Once the set time for handwashing begins, the timer 170 starts counting down. Upon completion of the predetermined duration, the speaker 172 is activated. The speaker 172 is configured to alert the user when the predetermined amount of time for handwashing is complete. This configuration is particularly useful in healthcare settings where proper hand hygiene is crucial for preventing the transmission of infectious diseases. By automating the timing and alerting processes, the compliance monitoring system 100 helps to standardize handwashing practices among all users, ensuring consistency and compliance, and reducing the overall risk of infection transmission within healthcare environments.

In another embodiment, the controlling mechanism 150 is configured to control the water source for handwashing, ensuring that the handwashing lasts for the predetermined duration. This embodiment may further comprise a motion sensor, thus identifying whether a user continues to wash their hands as the water remains on. If the motion sensor relays a deviation from the predetermined duration to the processor 102, the circuitry 104 triggers the controlling mechanism 150 to activate a barrier, or a locking system, which effectively restricts the user's movement, thereby preventing access to the next room until the user corrects the handwashing error and complies with the required PPE protocol.

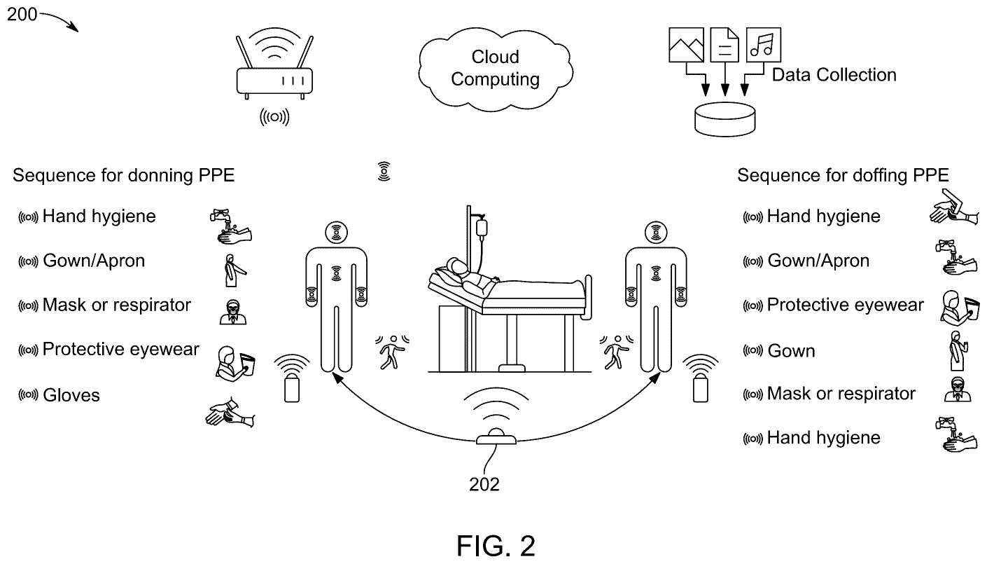

FIG. 2 illustrates one embodiment of the compliance monitoring system 100 for PPE items within a healthcare setting 200. The illustration depicts various components and their interconnections, highlighting capability of the compliance monitoring system 100 to ensure proper donning and doffing of the PPE items based on predetermined sequences stored in the memory 106. In one embodiment, a compliance analysis device 202 communicates wirelessly with a sensor reader, the sensor reader being used to read/scan individual PPE items equipped with sensors. The PPE items include the face mask 110 with the first sensor 114, the gown 120 with the second sensor 124, the face shield or eye shield 130 with the third sensor 134, and the righthand glove 142a and the lefthand glove 142b of the pair of gloves 140 with the fourth sensor 144a and the fifth sensor 144b. Each of these sensors are scanned by the sensor reader, the sensor reader then transmitting data, in the form of input signals, regarding the donning and doffing actions of the corresponding PPE item to the compliance analysis device 202, ensuring that each step is performed in accordance with the predetermined sequence.

The data collected from the input signals and other interactions within the compliance monitoring system 100 is sent to a cloud computing service, as shown in FIG. 2. Such cloud computing service, which may act as the processor 102, is responsible for further analysis, data storage, and sharing data across platforms for comprehensive compliance monitoring. The compliance monitoring system 100 may aggregate and analyze data regarding the use and compliance of PPE across different users and timeframes. Such analysis may be used for adjusting training programs, updating protocols, and improving overall safety measures within the healthcare facility.

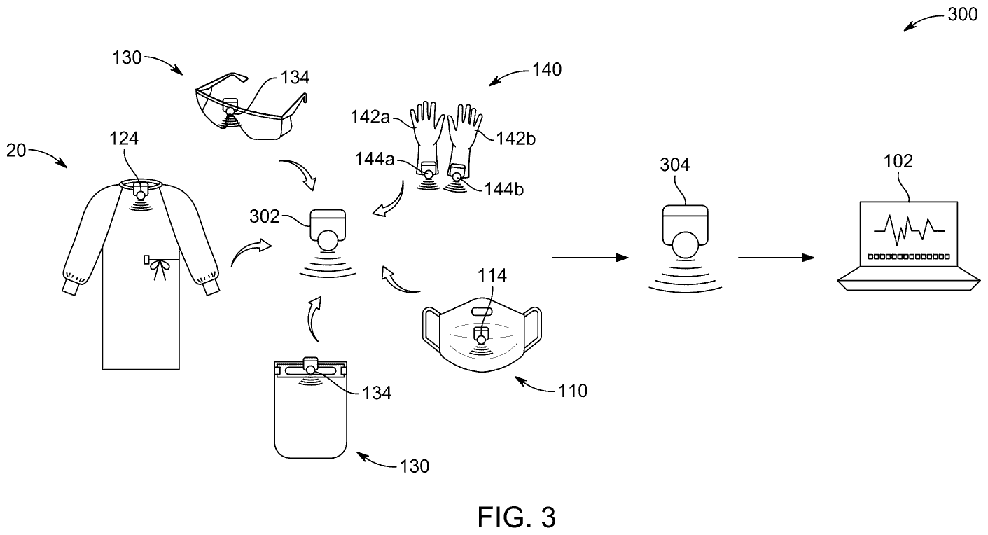

FIG. 3, illustrates a sequence detailing interaction and data flow of the compliance monitoring system, involving the sensors attached to various PPE items within the compliance monitoring system 100 (as generally represented by reference numeral 300). FIG. 3 sequentially depicts the various PPE components with the associated sensors, and the subsequent data transmission to the processor 102 when the sensors are read/scanned by the sensor reader, in the compliance monitoring system 100. The first sensor 114 within the face mask 110, the second sensor 124 within the gown 120, the third sensor 134 within the face shield or eye shield 130, and the fourth sensor 144a and the fifth sensor 144b within the righthand glove 142a and the lefthand glove 142b, respectively, of the pair of gloves 140, are scanned/read by the sensor reader to produce input signals, which are then communicated to the processor to detect specific interactions, such as donning or doffing, and transmit data corresponding to these interactions to a first data hub 302. Further, the aggregated data from the first data hub 302 may be transmitted to a second data hub 304, which may be in direct communication with the processor 102, which may be a computer or similar device. The processor 102 then may analyze this data and displays the output in a format that allows for real-time compliance analysis.

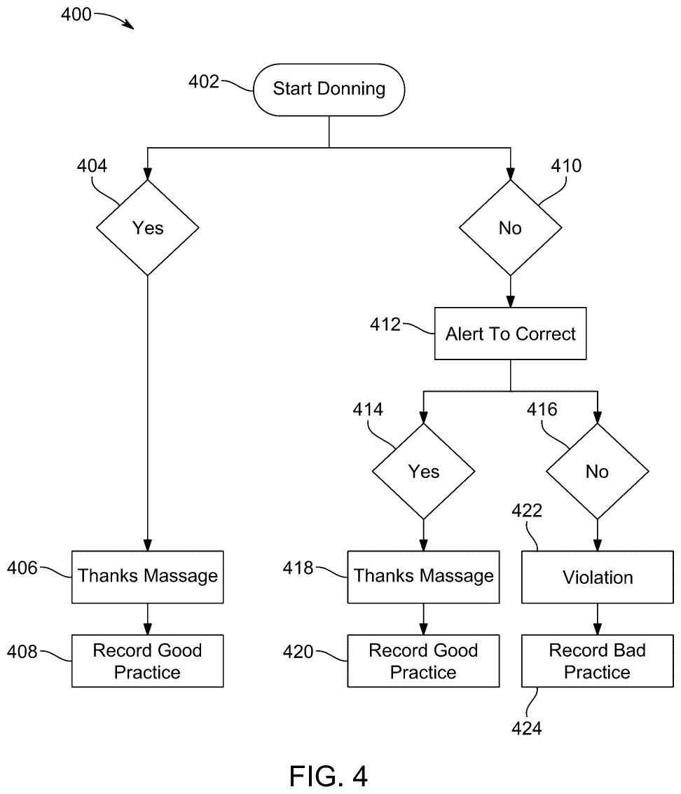

FIG. 4 illustrates a flowchart of a process 400 of monitoring donning procedures within the compliance monitoring system 100. At block 402, the process 400 begins with the user initiating the donning of PPE items. The system 100 begins to monitor the sequence of donning. At block 404, the system 100 checks if the donning sequence complies with the predetermined sequence stored in the memory 106 therein. If the user dons the PPE correctly according to the prescribed sequence, the process moves to block 406. At the block 406, the system 100 sends a ‘Thanks Message’ (for instance, to be displayed on the display device 160) as a positive reinforcement for following the correct procedures. Further, at block 408, alongside sending a thanks message, the system 100 records the instance as a good practice in the user's compliance history, stored in the memory 106. If the user dons the PPE incorrectly, as represented by block 410, the process 400 moves to block 412, where the system 100 issues an alert to the user to correct their actions. After the user has been alerted to correct their donning sequence, the system 100 checks again if the corrected sequence complies with the standards at blocks 414, 416. If, at the block 414, it is determined that the user has corrected the sequence successfully, the system sends a ‘Thanks Message’ at block 418 and record good practice at block 420. If, at the block 416, it is determined that the user is still following incorrect sequence, even after being alerted, the system 100 records this instance as a violation in the user's compliance history at block 422. At block 424, this record may trigger further actions such as additional training or other corrective measures. The process 400 outlines the compliance monitoring process within the system 100, ensuring adherence to proper PPE donning protocols through real-time monitoring and feedback mechanisms.

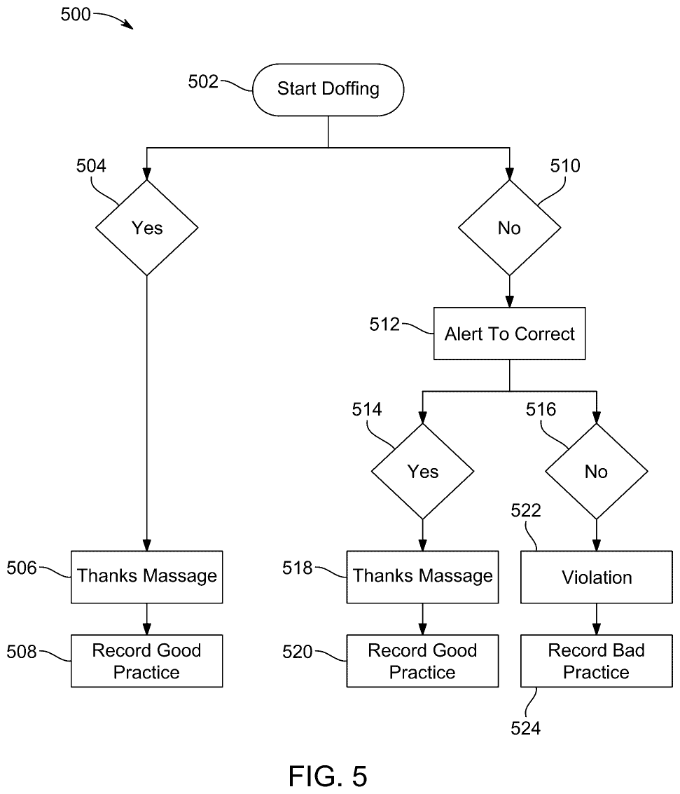

FIG. 5 illustrates a flowchart of a process 500 of monitoring doffing procedures within the compliance monitoring system 100. At block 502, the process 500 begins with the user initiating the doffing of PPE items. Herein, the system 100 begins to monitor the sequence of doffing. At block 504, the system 100 checks if the doffing sequence complies with the predetermined sequence stored in the memory 106 therein. If the user doffs the PPE correctly according to the prescribed sequence, the process moves to block 506. At the block 506, the system 100 sends a ‘Thanks Message’ (for instance, to be displayed on the display device 160) as a positive reinforcement for following the correct procedures. Further, at block 508, alongside sending a thanks message, the system 100 records the instance as a good practice in the user's compliance history, stored in the memory 106. If the user doffs the PPE incorrectly, as represented by block 510, the process 500 moves to block 512, where the system 100 issues an alert to the user to correct their actions. After the user has been alerted to correct their doffing sequence, the system 100 checks again if the corrected sequence complies with the standards at blocks 514, 516. If, at the block 514, it is determined that the user has corrected the sequence successfully, the system sends a ‘Thanks Message’ at block 518 and record good practice at block 520. If, at the block 516, it is determined that the user is still following incorrect sequence, even after being alerted, the system 100 records this instance as a violation in the user's compliance history at block 522. At block 524, this record may trigger further actions such as additional training or other corrective measures. The process 500 outlines the compliance monitoring process within the system 100, ensuring adherence to proper PPE doffing protocols through real-time monitoring and feedback mechanisms.

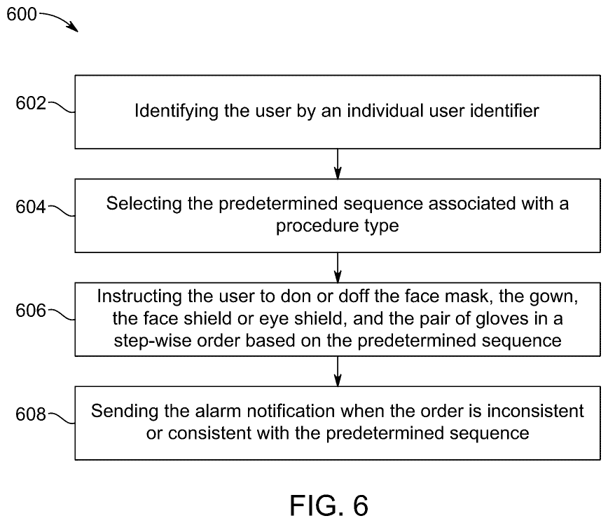

The present disclosure further provides a method of monitoring a user donning or doffing personal protective equipment with the compliance monitoring system 100. FIG. 6 illustrates a flowchart depicting steps involved in a method 600 of monitoring a user donning or doffing personal protective equipment with the compliance monitoring system 100, according to certain embodiments. These steps are only illustrative, and other alternatives can also be provided where one or more steps are added, one or more steps are removed, or one or more steps are provided in a different sequence without departing from the scope of the claims herein.

At step 602, the method 600 comprises identifying the user by an individual user identifier “U”. The individual user identifier “U” is unique to each user and is utilized by the system 100 to track and record the actions of the user throughout the donning or doffing process. The individual user identifier “U” ensures that all data collected is accurately attributed for compliance monitoring. At step 604, the method 600 comprises selecting the predetermined sequence associated with a procedure type. That is, once the user is identified, the system 100 selects the predetermined sequence that is associated with the specific type of procedure the user is preparing to undertake. This predetermined sequence is stored in the memory 106 of the system 100 and comprises detailed steps and sequence for how each PPE item should be worn or removed. The use of the predetermined sequence ensures that the user adheres to the specific requirements that are necessary for the safety and efficacy of the medical procedure to be performed.

At step 606, the method 600 comprises instructing the user to don or doff the face mask 110, the gown 120, the face shield or eye shield 130, and the pair of gloves 140 in a stepwise order based on the predetermined sequence. That is, after selecting the appropriate sequence, the system 100 instructs the user to begin donning or doffing the PPE in a stepwise order. This comprises the face mask 110, the gown 120, the face shield or eye shield 130, and the pair of gloves 140. Instructions can be delivered via the display device 160, which visually guides the user through each step, ensuring that the PPE is worn or removed in the correct sequence. At step 608, the method 600 comprises sending the alarm notification when the order is inconsistent or consistent with the predetermined sequence. As the user dons or doffs the PPE, the user scans/reads the sensors attached to each PPE item with the sensor reader, the sensor reader then transmitting input signals to the processor 102 associated with each sensor read/scanned. The system 100 evaluates these signals to determine if the order of application or removal matches the predetermined sequence. If any deviation is detected, the circuitry 104 sends the alarm notification to the user to alert them of the inconsistency. Conversely, if the sequence is followed correctly, a notification may also be sent to confirm correct compliance, reinforcing proper PPE usage.

In an embodiment, the method 600 further comprises individually scanning the first through fifth sensor before the user dons or doffs the face mask 110, the gown 120, the face shield or eye shield 130, and the pair of gloves 140. That is, prior to the start of the donning or doffing process, each sensor, namely the first sensor 114 on the face mask 110, the second sensor 124 on the gown 120, the third sensor 134 on the face shield or eye shield 130, and the fourth sensor 144a and fifth sensor 144b on the gloves 140, is individually scanned by the sensor reader.

In another embodiment of the compliance monitoring system 100 for donning compliance, sensors are assigned to different PPE items and related hygiene stations to ensure proper adherence to donning protocols. Specifically, sensor s1 is connected to the handwashing sink, sensor s2 to the gown 120, sensor s3 to the face mask 110, sensor s4 to the protective eyewear 130, and sensor s5 to the gloves 140. Each sensor is activated when the user reads/scans the sensor associated contact with the corresponding PPE item or hygiene station with the sensor reader. Upon activation, the sensor reader sends an input signal to the processor 102 comprising a timestamp. These timestamps, designated as ts1, ts2, ts3, ts4, and ts5 for sensors s1, s2, s3, s4, and s5 respectively, are used by the processor 102 to determine the exact order in which the PPE items were donned. Herein, the predetermined sequence for donning the PPE items, as stored in the memory 106, defines the following order: performing hand hygiene (ts1), and donning the gown 120 (ts2), the face mask 110 (ts3), the protective eyewear 130 (ts4), and the gloves 140 (ts5). The processor 102 compares the received timestamps against this sequence. If the timestamps reflect that the PPE was donned in the correct sequence (ts1<ts2<ts3<ts4<ts5), the processor 102 generates a notification indicating “Good Practice”. Conversely, if the timestamps reveal any deviation from the established order, a notification of “Bad Practice” is issued. The complete logic for the donning sequence (donning algorithm 1) is given as follows: input: ts1, ts2, ts3, ts4, ts5

output: Record of Good or Bad practice

| | |
|---|---|
| 1 | if ts1 < ts2 < ts3 < ts4 < ts5 then |
| 2 | \| Record as “Good Practice” |
| 3 | else |
| 4 | \| Send alert to medical personnel |
| 5 | // Update timestamps based on persons new PPE insertion order: |
| 6 | ts1 = ts1new |
| 7 | ts2 = ts2new |
| 8 | ts3 = ts3new |
| 9 | ts4 = ts4new |
| 10 | ts5 = ts5new |
| 11 | if ts1 < ts2 < ts3 < ts4 < ts5 then |
| 12 | \| Record as “Good Practice” |
| 13 | else |
| 14 | \| Record as “Bad Practice” |
| 15 | end |

The above donning algorithm describes the process for evaluating the sequence of donning PPE and issuing a compliance status based on the timestamps of each action. This ensures that each step in the PPE donning process is performed in accordance with established health and safety protocols, enhancing the compliance and safety measures within the healthcare facility.

In another embodiment of the compliance monitoring system 100 for doffing compliance, sensors are assigned to different PPE items and related hygiene stations to ensure proper adherence to doffing protocols. Specifically, sensor s1 is connected to the handwashing sink, sensor s2 to the gown 120, sensor s3 to the face mask 110, sensor s4 to the protective eyewear 130, and sensor s5 to the gloves 140. Each sensor is activated when the user reads/scans the sensor associated contact with the corresponding PPE item or hygiene station with the sensor reader. Upon activation, the sensor reader sends an input signal to the processor 102 comprising a timestamp. These timestamps, designated as ts1, ts2, ts3, ts4, and ts5 for sensors s1, s2, s3, s4, and s5 respectively, are used by the processor 102 to determine the exact order in which the PPE items were doffed. The predetermined sequence for doffing the PPE items, as stored in the memory 106, defines the following order: removing the gloves 140 (ts5), the protective eyewear 130 (ts4), the face mask 110 (ts3), the gown 120 (ts2), and performing hand hygiene (ts1). The processor 102 compares the received timestamps against this sequence. If the timestamps reflect that the PPE was doffed in the correct sequence (ts5<ts4<ts3<ts2<ts1), the processor 102 generates a notification indicating “Good Practice”. Conversely, if the timestamps reveal any deviation from the established order, a notification of “Bad Practice” is issued. The complete logic for the doffing sequence (doffing algorithm 2) is given as follows:

input: ts1a, ts1b, ts2, ts3, ts4, ts5
output: Record of Good or Bad practice

| | |
|---|---|
| 1 | if ts5 < ts1a < ts4 < ts2 < ts3 < ts1b, then |
| 2 | \| Record as “Good Practice” |
| 3 | else |
| 4 | \| Send alert to medical personnel |
| 5 | // Update timestamps based on persons new PPE removal order: |
| 6 | ts1a = ts1newa |
| 7 | ts1b = ts1newb |
| 8 | ts2 = ts2new |
| 9 | ts3 = ts3new |
| 10 | ts4 = ts4new |
| 11 | ts5 = ts5new |
| 12 | if ts5 < ts1a < ts4 < ts2 < ts3 < ts1b then |
| 13 | \| Record as “Good Practice” |
| 14 | else |
| 15 | \| Record as “Bad Practice” |
| 16 | end |

The above doffing algorithm describes the process for evaluating the sequence of doffing PPE and issuing a compliance status based on the timestamps of each action. This ensures that each step in the PPE doffing process is performed in accordance with established health and safety protocols, enhancing the compliance and safety measures within the healthcare facility.

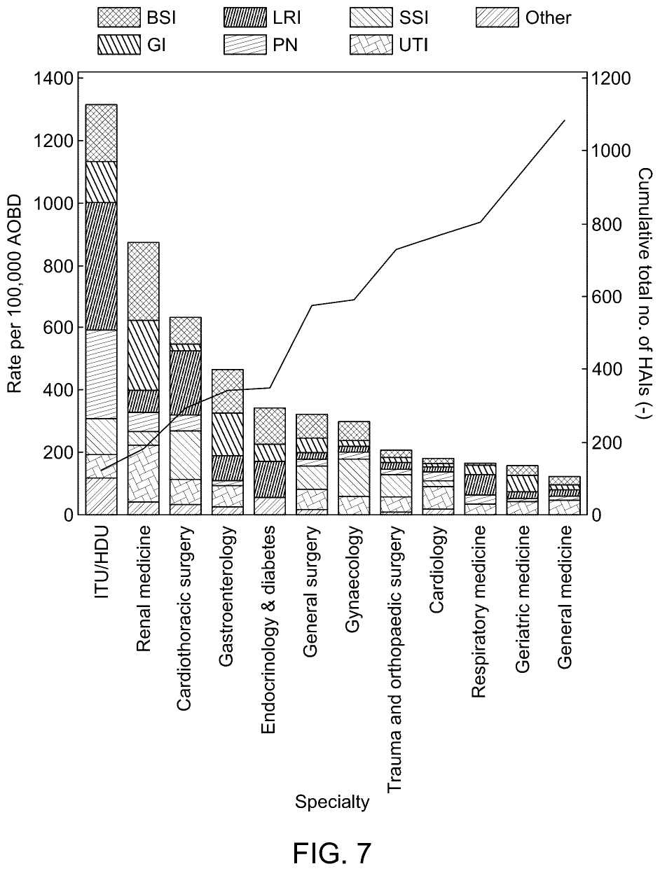

FIG. 7 illustrates the incidence of healthcare-associated infections (HAIs) by medical specialty and the distribution of HAI types within each specialty for the year 2022. The horizontal axis categorizes various medical specialties such as intensive therapy unit (ITU/HDU), renal medicine, cardiology, surgery, and others. Each bar on the graph represents the rate of HAIs per 100,000 acute occupied bed-days (AOBD), broken down by infection type including bloodstream infection (BSI), gastrointestinal infection (GI), lower respiratory tract infection (LRI), pneumonia (PN), surgical site infection (SSI), urinary tract infection (UTI), and other types of infections. The accompanying line graph shows the cumulative total of HAIs across all specialties, indicating trends over time or across specialties.

The graph provides the significance of stringent infection prevention measures, including the proper use and compliance with personal protective equipment (PPE) protocols. As shown, there are some specific areas where HAIs are most prevalent, and thus healthcare facilities can target their infection control efforts more effectively, focusing on specialties that show higher rates of infection and potentially adjusting protocols to address these challenges. The compliance monitoring system 100 of the present disclosure can be implemented to enhance these efforts by ensuring the correct sequence of PPE donning and doffing is followed. The compliance monitoring system 100 of the present disclosure utilizes a sensor reader and sensors associated unique identifiers integrated into various items of PPE items. The system 100 is configured to monitor the sequence of donning and doffing PPE in real-time, using a predetermined sequence stored in the memory 106 of the processor 102. In contrast to known systems, which largely relies on manual checks or intermittent compliance assessments, the compliance monitoring system 100 automates this process, thereby reducing human error and increasing the reliability of compliance monitoring in healthcare settings.

The compliance monitoring system 100 is further capable of providing immediate feedback to users through a combination of visual and auditory alerts from the display device 160 and speaker 172, respectively. If a deviation from the predetermined sequence is detected, the system 100 notifies the user to correct their procedure and records this event in the memory 106 for tracking compliance trends and identifying areas for improvement. The compliance monitoring system 100 helps in systematically reducing the incidence of non-compliance and providing actionable insights into PPE usage, thereby providing a safer working environment for healthcare professionals, and enhancing the overall quality of patient care.

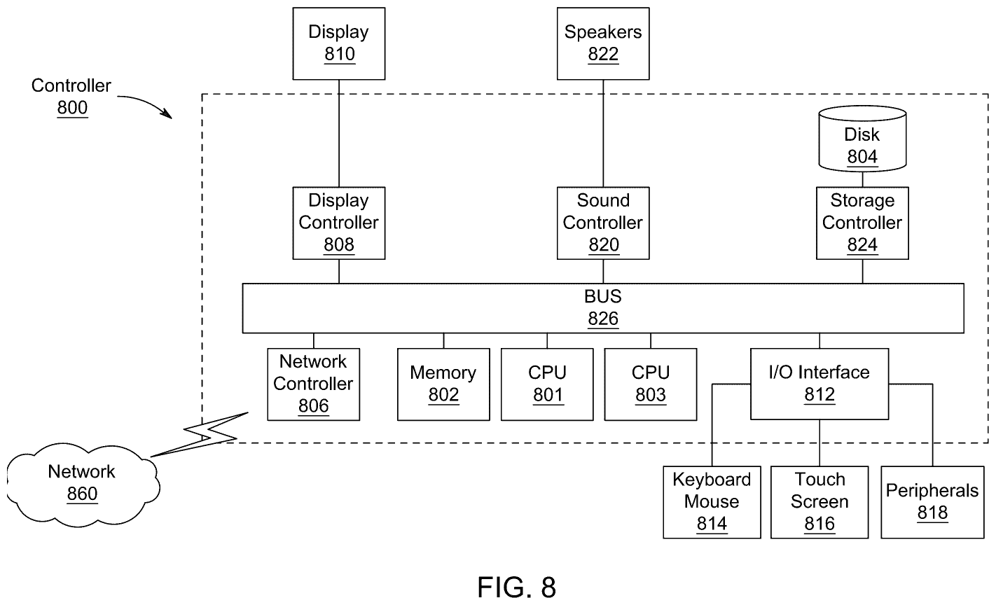

Next, further details of the hardware description of the computing environment according to exemplary embodiments is described with reference to FIG. 8. In FIG. 8, a controller 800 is described which may be utilized to control operations of various components of the system 100, 200, 300 of the present disclosure, in which the controller is a computing device which comprises a central processing unit (CPU) 801 which performs the processes described above/below. The process data and instructions may be stored in memory 802. These processes and instructions may also be stored on a storage medium disk 804 such as a hard drive (HDD) or portable storage medium or may be stored remotely.

Further, the claims are not limited by the form of the computer-readable media on which the instructions of the inventive process are stored. For example, the instructions may be stored on compact discs (CDs), digital video disc (DVDs), in FLASH memory, random access memory (RAM), read-only memory (ROM), programmable read-only memory (PROM), erasable programmable read-only memory (EPROM), electronically erasable programmable read-only memory (EEPROM), hard disk or any other information processing device with which the computing device communicates, such as a server or computer.

Further, the claims may be provided as a utility application, background daemon, or component of an operating system, or combination thereof, executing in conjunction with CPU 801, 803 and an operating system such as Microsoft Windows 7, Microsoft Windows 8, Microsoft Windows 10, UNIX, Solaris, LINUX, Apple MAC-OS, and other systems known to those skilled in the art.

The hardware elements to achieve the computing device may be realized by various circuitry elements, known to those skilled in the art. For example, CPU 801 or CPU 803 may be a Xenon or Core processor or may be other processor types that would be recognized by one of ordinary skill in the art. Alternatively, the CPU 801, 803 may be implemented on an FPGA, ASIC, PLD or using discrete logic circuits, as one of ordinary skill in the art would recognize. Further, CPU 801, 803 may be implemented as multiple processors cooperatively working in parallel to perform the instructions of the inventive processes described above.

The computing device in FIG. 8 also comprises a network controller 806 for interfacing with network 860. As can be appreciated, the network 860 can be a public network, such as the Internet, or a private network such as a local area network (LAN) or a wide-area network (WAN), or any combination thereof and can also include a public switched telephone network (PSTN) or an integrated services digital network (ISDN). The network 860 can also be wired, such as an Ethernet network, or can be wireless such as a cellular network including EDGE, 3G, 4G and 5G wireless cellular systems. The wireless network can also be Wi-Fi, Bluetooth, or any other wireless form of communication that is known.

The computing device further comprises a display controller 808, such as a NVIDIA GeForce GTX or Quadro graphics adaptor for interfacing with display 810, such as an LCD monitor. A general-purpose input-output (I/O) interface 812 interfaces with a keyboard and/or mouse 814 as well as a touch screen panel 816 on or separate from display 810. A general purpose I/O interface may also connect a variety of peripherals 818 including printers and scanners.

A sound controller 820 is also provided in the computing device to interface with speakers/microphone 822 thereby providing sounds and/or music.

The general-purpose storage controller 824 connects the storage medium disk 804 with a communication bus 826, which may be an instruction set architecture (ISA) bus, an extended industry standard architecture (EISA) bus, a video electronics standards association (VESA) bus, a peripheral component interconnect (PCI) bus, or similar, for interconnecting all of the components of the computing device. A description of the general features and functionality of the display 810, keyboard and/or mouse 814, as well as the display controller 808, storage controller 824, network controller 806, sound controller 820, and general purpose I/O interface 812 is omitted herein as these features are known in the art.

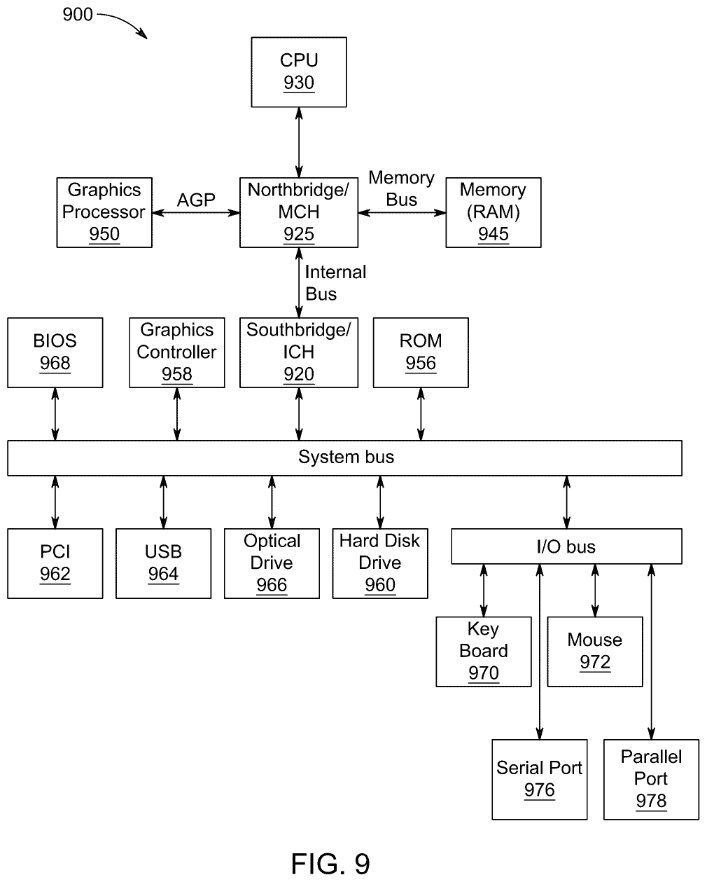

The exemplary circuit elements described in the context of the present disclosure may be replaced with other elements and structured differently than the examples provided herein. Moreover, circuitry configured to perform features described herein may be implemented in multiple circuit units (e.g., chips), or the features may be combined in circuitry on a single chipset, as shown on FIG. 9.

FIG. 9 shows a schematic diagram of a data processing system, according to certain embodiments, for performing the functions of the exemplary embodiments. The data processing system is an example of a computer in which code or instructions implementing the processes of the illustrative embodiments may be located.

In FIG. 9, data processing system 900 employs a hub architecture comprising a north bridge and memory controller hub (NB/MCH) 925 and a south bridge and input/output (I/O) controller hub (SB/ICH) 920. The CPU 930 is connected to NB/MCH 925. The NB/MCH 925 also connects to the memory 945 via a memory bus and connects to the graphics processor 950 via an accelerated graphics port (AGP). The NB/MCH 925 also connects to the SB/ICH 920 via an internal bus (e.g., a unified media interface or a direct media interface). The CPU 930 may contain one or more processors and even may be implemented using one or more heterogeneous processor systems.

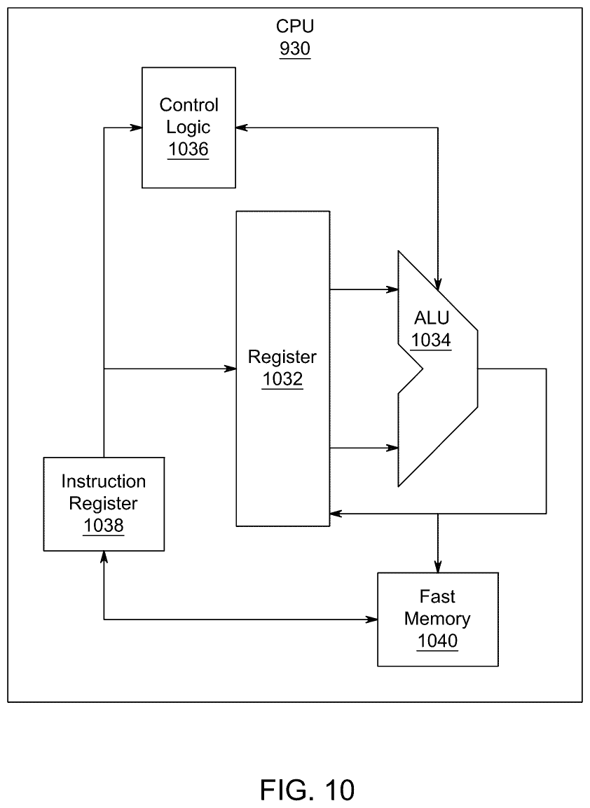

For example, FIG. 10 shows one implementation of CPU 930. In one embodiment, the instruction register 1038 retrieves instructions from the fast memory 1040. At least part of these instructions is fetched from the instruction register 1038 by the control logic 1036 and interpreted according to the instruction set architecture of the CPU 930. Part of the instructions can also be directed to the register 1032. In one implementation the instructions are decoded according to a hardwired method, and in another implementation the instructions are decoded according to a microprogram that translates instructions into sets of CPU configuration signals that are applied sequentially over multiple clock pulses. After fetching and decoding the instructions, the instructions are executed using the arithmetic logic unit (ALU) 1034 that loads values from the register 1032 and performs logical and mathematical operations on the loaded values according to the instructions. The results from these operations can be feedback into the register and/or stored in the fast memory 1040. According to certain implementations, the instruction set architecture of the CPU 930 can use a reduced instruction set architecture, a complex instruction set architecture, a vector processor architecture, a very large instruction word architecture. Furthermore, the CPU 930 can be based on the Von Neuman model or the Harvard model. The CPU 930 can be a digital signal processor, a field programmable gate array processor (FPGA), an application-specific integrated circuit (ASIC) processor, a programmable logic array (PLA) circuit, a programmable logic device (PLD), or a complex programmable logic device (CPLD). Further, the CPU 930 can be an x86 processor, an ARM processor, a power architecture processor, a scalable processor architecture (SPARC) processor, or another known CPU architecture.

Referring again to FIG. 9, the data processing system 900 can include that the SB/ICH 920 is coupled through a system bus to an I/O Bus, a read only memory (ROM) 956, universal serial bus (USB) port 964, a flash binary input/output system (BIOS) 968, and a graphics controller 958. PCI/PCIe devices can also be coupled to SB/ICH 988 through a PCI bus 962.

The PCI devices may include, for example, Ethernet adapters, add-in cards, and PC cards for notebook computers. The hard disk drive 960 and CD-ROM 966 can use, for example, an integrated drive electronics (IDE) or serial advanced technology attachment (SATA) interface. In one implementation the I/O bus can include a super I/O (SIO) device.

Further, the hard disk drive (HDD) 960 and optical drive 966 can also be coupled to the SB/ICH 920 through a system bus. In one implementation, a keyboard 970, a mouse 972, a parallel port 978, and a serial port 976 can be connected to the system bus through the I/O bus. Other peripherals and devices that can be connected to the SB/ICH 920 using a mass storage controller such as SATA or PATA, an Ethernet port, an ISA bus, a LPC bridge, SMBus, a DMA controller, and an Audio Codec.

Moreover, the present disclosure is not limited to the specific circuit elements described herein, nor is the present disclosure limited to the specific sizing and classification of these elements. For example, the skilled artisan will appreciate that the circuitry described herein may be adapted based on changes on battery sizing and chemistry or based on the requirements of the intended back-up load to be powered.

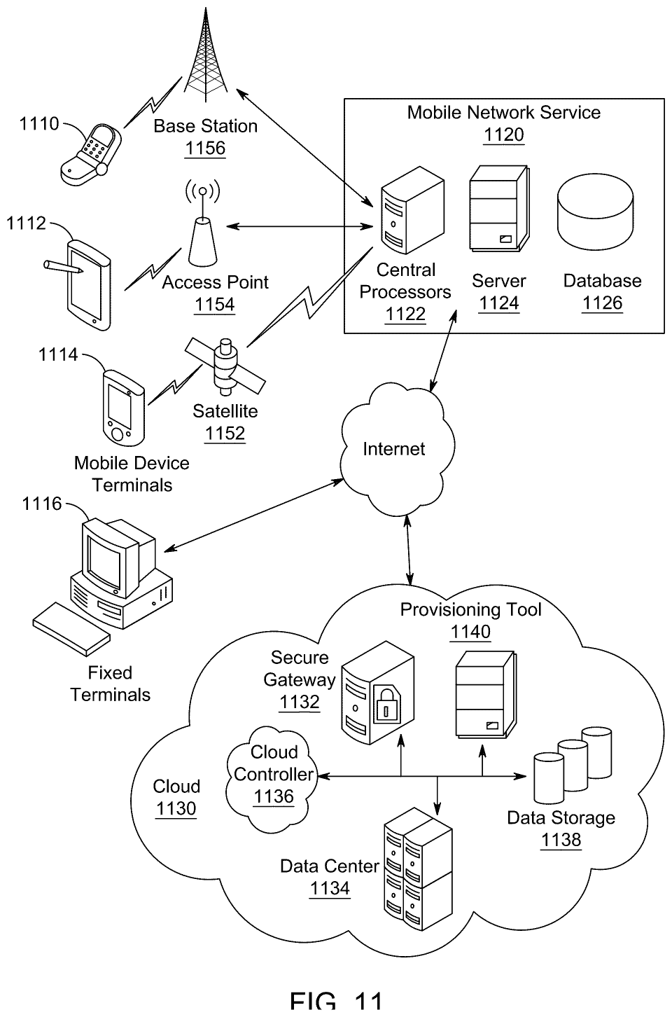

The functions and features described herein may also be executed by various distributed components of a system. For example, one or more processors may execute these system functions, wherein the processors are distributed across multiple components communicating in a network. The distributed components may include one or more client and server machines, such as cloud 1130 including a cloud controller 1136, a secure gateway 1132, a data center 1134, data storage 1138 and a provisioning tool 1140, and mobile network services 1120 comprising central processors 1122, a server 1124 and a database 1126, which may share processing, as shown by FIG. 11, in addition to various human interface and communication devices (e.g., display monitors 1116, smart phones 1110, tablets 1112, personal digital assistants (PDAs) 1114). The network may be a private network, such as a LAN, satellite 1152 or WAN 1154, or be a public network, may such as the Internet. Input to the system may be received via direct user input and received remotely either in real-time or as a batch process. Additionally, some implementations may be performed on modules or hardware not identical to those described. Accordingly, other implementations are within the scope that may be claimed.

The above-described hardware description is a non-limiting example of corresponding structure for performing the functionality described herein.

Numerous modifications and variations of the present disclosure are possible in light of the above teachings. It is therefore to be understood that within the scope of the appended claims, the invention may be practiced otherwise than as specifically described herein.

1. A compliance monitoring system, comprising:
a face mask having a first unique identifier and comprising a first sensor placed on a middle section of the face mask, wherein the middle section is configured to be positioned near a mouth of a user;
a gown having a second unique identifier and comprising a second sensor disposed on a neckline of the gown;
a face shield or an eye shield having a third unique identifier and comprising a third sensor disposed on an upper forehead section of the face shield or a nose bridge of the eye shield;
a pair of gloves having a forth and fifth unique identifiers, respectively, and comprising a fourth sensor and a fifth sensor individually disposed on a righthand and a lefthand of the pair of gloves; and
a sensor reader in wireless communication with a processor,
wherein the processor having circuitry and a memory,
wherein the circuitry includes instructions configured to:
receive input signals from the sensor reader, and
send a first alarm notification when the face mask, the gown, the face shield or eye shield, and the pair of gloves are donned or doffed by a user in an order that is either consistent or inconsistent with a predetermined sequence saved in the memory of the processor,
wherein the circuitry also includes instructions configured to record input signals from the first, second, third, fourth, and/or fifth sensor and/or the corresponding unique identifier and send a second alarm notification when the face mask, the gown, the face shield or eye shield or the pair of gloves are not designated for a specific medical procedure or environment.

2. The system of claim 1, wherein each sensor is an RFID tag.

3. The system of claim 1,
wherein each sensor with the paired unique identifier is activated when scanned by the sensor reader as the user dons the face mask, the gown, the face shield or eye shield, or the pair of gloves.

4. The system of claim 1, wherein the memory is configured to store doffing and donning compliance data for a plurality of users, and
wherein the circuitry is further configured to determine a compliance rate of the plurality of users based on the doffing and donning compliance data.

5. The system of claim 1, wherein each of the face mask, gown, the face shield or eye shield and the pair of gloves further comprises a color sensor.

6. (canceled)

7. The system of claim 1, wherein each user is associated with an individual user identifier, and
wherein the memory of the processor is further configured to store a plurality of the individual user identifiers.

8. The system of claim 1, wherein the predetermined sequence is individually coded for a procedure type based on a selected compliance standard.

9. The system of claim 1, further comprising:
a controlling mechanism,
wherein the system is disposed in a first room and the controlling mechanism is configured to stop the user from proceeding into a next room from the first room when the order is inconsistent with the predetermined sequence.

10. The system of claim 1, wherein the circuitry is further configured to alert the user to stop and correct the order to be consistent with the predetermined sequence before proceeding when the order is inconsistent with the predetermined sequence.

11. The system of claim 1, wherein each sensor is an RFID tag.

12. The system of claim 1, wherein the sensor reader is worn by or attached to the user.

13. The system of claim 12, wherein when the monthly average of noncompliance exceeds a predetermined limit, a notification is created to alert the user an additional training program is needed.

14. The system of claim 1, further comprising:
a display device,
wherein the display device is configured to show an image of the face mask, the gown, the face shield or eye shield, and the pair of gloves in the order consistent with the predetermined sequence.

15. The system of claim 14, wherein the display device is configured to show a video of a model user donning or doffing the face mask, the gown, the face shield or eye shield, and the pair of gloves in the order consistent with the predetermined sequence.

16. The system of claim 1, further comprising:
a timer; and
a speaker,
wherein the timer is set for a predetermined amount of time for handwashing when required by the predetermined sequence, and
wherein the speaker is configured to alert the user when the predetermined amount of time for handwashing is complete.

17. The system of claim 1, wherein the memory is further configured to store a cleaning history such that when the system is placed in a clean room for sterile compounding, the system is configured to alert the user that a cleaning is due, consistent with a predetermined clean room standard.

18. The system of claim 17, further comprising:
a cleaning sensor selected from the group consisting of a serial number, a barcode, an integrated chip, or an RFID tag,
wherein when the cleaning is complete, the circuitry is configured to receive an input signal from the sensor reader when the cleaning sensor is scanned to create a cleaning log; and
wherein the memory is configured to store a plurality of the cleaning logs.

19. A method of monitoring a user donning or doffing personal protective equipment with the system of claim 1, comprising:
identifying the user by an individual user identifier;
selecting the predetermined sequence associated with a procedure type;
instructing the user to don or doff the face mask, the gown, the face shield or eye shield, and the pair of gloves in a stepwise order based on the predetermined sequence; and
sending the alarm notification when the order is inconsistent or consistent with the predetermined sequence.

20. The method of claim 19, further comprising:
individually scanning the first through fifth sensor with the sensor reader before the user dons or doffs the face mask, the gown, the face shield or eye shield, and the pair of gloves.

21. The system of claim 1, wherein the unique identifier is one or more selected from the group consisting of a serial number, a barcode, an integrated chip, and a radio frequency identification (RFID) tag.
